(19) United States
(12) Patent Application Publication
(10) Pub. No.: US 2026/0337168 A1
(43) Pub. Date: Oct. 29, 2026

(54) METHOD FOR PRODUCING BENZOXAZINE COMPOUND

(71) Applicant: HONSHU CHEMICAL INDUSTRY CO., LTD., Chuo-ku, Tokyo (JP)

(72) Inventors: Kenta HAGIWARA, Wakayama-shi (JP); Takahiro ASAEDA, Wakayama-shi (JP)

(21) Appl. No.: 19/108,678

(22) PCT Filed: Nov. 17, 2023
(86) PCT No.: PCT/JP2023/041386

§ 371 Date: Mar. 4, 2025

(30) Foreign Application Priority Data
Nov. 17, 2022 (JP) 2022-183990

(51) Int. Cl. C07D 265/16

(52) U.S. Cl. CPC C07D 265/16

(57) ABSTRACT

An object is to provide a method for producing a highly pure benzoxazine compound by suppressing a decrease in purity due to an increase in molecular weight during the production of an allyl group-containing benzoxazine compound. As a solution, a method for producing a benzoxazine compound represented by general formula (1), including a solvent distilling-off step of subjecting a benzoxazine solution containing the benzoxazine compound represented by general formula (1) and an ester solvent having a boiling point at 1 atm in the range of 30 °C or higher and 90 °C or lower to removal of the solvent by distillation in the range of 20 °C or higher and 85 °C or lower, is provided.

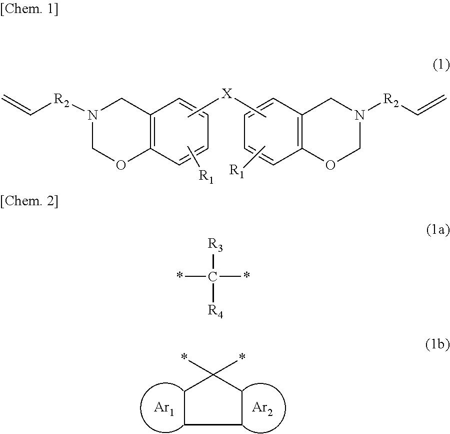

### TECHNICAL FIELD

The present invention relates to a method for producing a benzoxazine compound. The invention particularly relates to a method for producing a benzoxazine compound having benzoxazine rings at both ends of a linking group and further having an unsaturated group.

### BACKGROUND ART

Benzoxazine compounds, which are compounds synthesized by reacting a phenol, an amine, and formaldehyde, are known as thermosetting resin raw materials that, when heated, undergo ring-opening polymerization of a benzoxazine ring to cure without producing any volatile by-products, and are used as raw materials of molded bodies usable as materials for insulating substrates, liquid crystal alignment agents, resin compositions for semiconductor sealing, and the like. In these applications, stability at high temperatures and highly reliable heat resistance are required.

On the other hand, benzoxazine compounds have drawbacks in that, in general, their curing temperatures are relatively high and their cured products are brittle. In addition, even higher heat resistance is also expected. In recent years, to improve this point, benzoxazine compounds in which cross-linkable functional groups are introduced have been developed. In particular, it has been reported that a cured product of a benzoxazine resin in which an allyl group is introduced has high heat resistance (PTL 1).

Examples of known methods for synthesizing a benzoxazine compound having an allyl group include a method in which allylamine is dissolved in dioxane, a formaldehyde liquid is added dropwise to cause a reaction at room temperature, and then bisphenol A is added to cause a reaction at 100 °C to 120 °C (PTL 1), a method in which bisphenol F, allylamine, and paraformaldehyde as raw materials are mixed at one time together with a solvent to cause a reaction (NPL 1), and a method in which after allylamine hydrochloride is added to an aqueous sodium hydroxide solution, paraformaldehyde is added to cause a reaction at room temperature, and then bisphenol A and toluene as a solvent are added to cause a reaction at 100 °C (PTL 2).

### CITATION LIST

### Patent Literature

PTL 1: Japanese Unexamined Patent Application Publication No. 2003-286320
PTL 2: Chinese Patent Application Publication No. 111138613

### Non Patent Literature

NPL 1: Polymer International 2012, Vol. 62, p. 966-973

### SUMMARY OF INVENTION

### Technical Problem

Regarding the above methods for producing an allyl group-containing benzoxazine compound known in the art, the method described in PTL 2 has problems in that since the reaction is carried out at a temperature of 100 °C, and the use of toluene as a reaction solvent necessitates a high temperature of 90 °C or higher for solvent distilling-off performed after completion of the reaction and also for the removal of contained water, during the reaction and the post-reaction treatment, polymerization is initiated due to heat to decrease the purity of the benzoxazine compound, and as the polymerization proceeds, the benzoxazine compound becomes more viscous and less easy to handle. In the method described in PTL 1, the reaction is carried out at a temperature of 100 °C to 120 °C, and in addition, the use of dioxane as a solvent necessitates a high temperature for solvent distilling-off as in the case of toluene and also creates difficulty in handling in terms of wastewater treatment.

An object of the present invention is to provide a method for producing a highly pure benzoxazine compound by suppressing a decrease in purity due to an increase in molecular weight during the production of a benzoxazine compound having an unsaturated group.

### Solution to Problem

To achieve the above object, the present inventors have conducted intensive studies and found that the above object can be achieved by preparing a solution of a benzoxazine compound having an unsaturated group and a specific solvent and removing the solvent in a specific temperature range, thereby completing the present invention.

The present invention is as follows.

1. A method for producing a benzoxazine compound represented by general formula (1), including a solvent distilling-off step of subjecting a benzoxazine solution containing the benzoxazine compound represented by general formula (1) and an ester solvent having a boiling point at 1 atm in a range of 30 °C or higher and 90 °C or lower to removal of the solvent by distillation in a range of 20 °C or higher and 85 °C or lower.

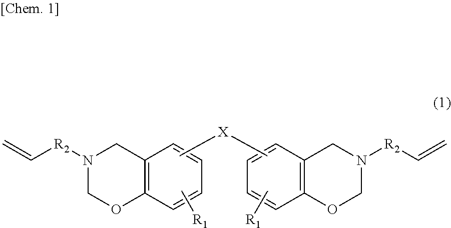

(In the formula, each R₁ independently represents a hydrogen atom or an alkyl group having 1 to 6 carbon atoms, each R₂ independently represents a divalent hydrocarbon group having 1 to 10 carbon atoms, and X represents a single bond, an oxygen atom, a sulfur atom, a sulfonyl group, a carbonyl group, a divalent group represented by general formula (1a), or a divalent group represented by (1b).)

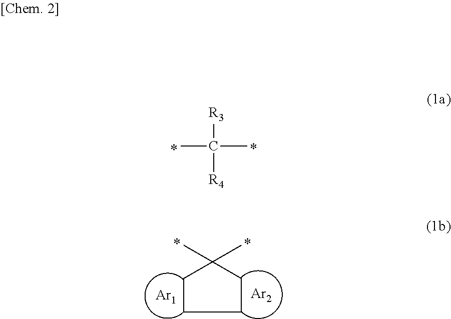

(In general formulas (1a) and (1b), R₃ and R₄ each independently represent a hydrogen atom, an alkyl group having 1 to 10 carbon atoms, an alkyl halide group having 1 to 10 carbon atoms, or an aryl group having 6 to 12 carbon atoms, R₃ and R₄ may be bonded to each other to together form a cycloalkylidene group having 5 to 20 carbon atoms, Ar₁ and Ar₂ each independently represent an aryl group having 6 to 12 carbon atoms, and each * represents a bonding position.)
2. The production method according to 1., wherein the ester solvent is ethyl acetate.
3. The production method according to 1., wherein the benzoxazine compound represented by general formula (1) is at least one compound selected from compounds (1-1) to (1-6).

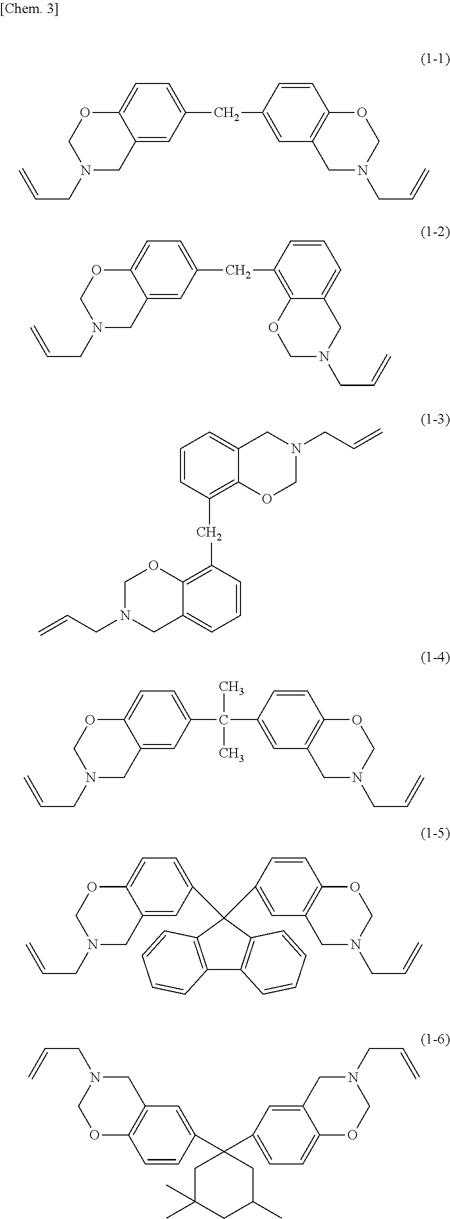

4. The production method according to 1., wherein the benzoxazine solution is a benzoxazine solution obtained through a reaction step of performing a reaction in the presence of a bisphenol compound represented by general formula (2), a formaldehyde, an amine compound represented by general formula (3), and the ester solvent having a boiling point at 1 atm in the range of 30 °C or higher and 90 °C or lower.

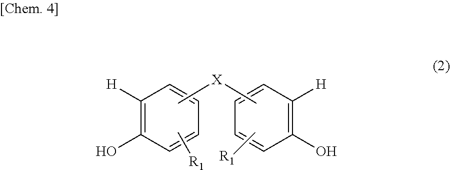

(In the formula, R₁ and X are as defined in general formula (1).)

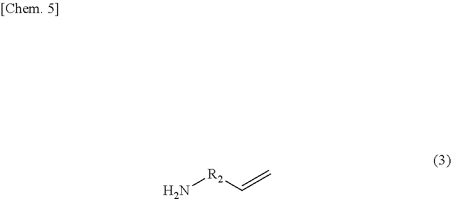

(In the formula, R₂ is as defined in general formula (1).)
5. The production method according to 4., wherein the bisphenol compound represented by general formula (2) is at least one compound selected from bisphenol F (bis(2-hydroxyphenyl)methane, 2-hydroxyphenyl-4-hydroxyphenylmethane, bis(4-hydroxyphenyl)methane), bisphenol A (2,2-bis(4-hydroxyphenyl)propane), bisphenol C (2,2-bis(4-hydroxy-3-methylphenyl)propane), bisphenol Z (1,1-bis(4-hydroxyphenyl)cyclohexane), bisphenol TMC (1,1-bis(4-hydroxyphenyl)-3,3,5-trimethylcyclohexane), and 9,9-bis(4-hydroxyphenyl)fluorene, and the amine compound represented by general formula (3) is allylamine.

### Advantageous Effects of Invention

According to the method for producing a benzoxazine compound according to the present invention, a benzoxazine compound having an unsaturated group with improved purity can be produced by suppressing the formation of compounds having molecular weights higher than that of the benzoxazine compound (hereinafter also referred to simply as “high-molecular-weight components”).

### DESCRIPTION OF EMBODIMENTS

### <Method for Producing Benzoxazine Compound According to Present Invention>

A method for producing a benzoxazine compound represented by general formula (1) according to the present invention is a production method including a solvent distilling-off step of subjecting a benzoxazine solution containing the benzoxazine compound represented by general formula (1) and an ester solvent having a boiling point at 1 atm in the range of 30 °C or higher and 90 °C or lower to removal of the solvent by distillation in the range of 20 °C or higher and 85 °C or lower.

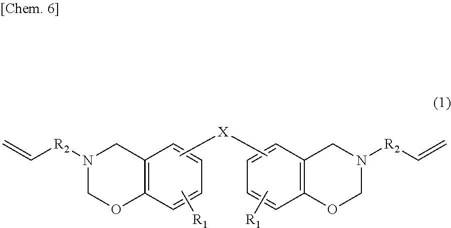

(In the formula, each R₁ independently represents a hydrogen atom or an alkyl group having 1 to 6 carbon atoms, each R₂ independently represents a divalent hydrocarbon group having 1 to 10 carbon atoms, and X represents a single bond, an oxygen atom, a sulfur atom, a sulfonyl group, a carbonyl group, a divalent group represented by general formula (1a), or a divalent group represented by (1b).)

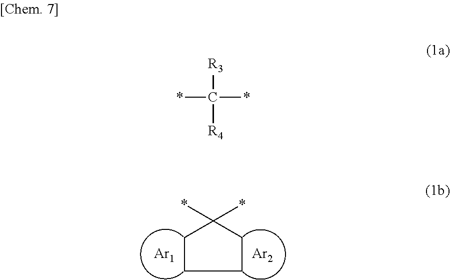

(In general formulas (1a) and (1b), R₃ and R₄ each independently represent a hydrogen atom, an alkyl group having 1 to 10 carbon atoms, an alkyl halide group having 1 to 10 carbon atoms, or an aryl group having 6 to 12 carbon atoms, R₃ and R₄ may be bonded to each other to together form a cycloalkylidene group having 5 to 20 carbon atoms, Ar₁ and Ar₂ each independently represent an aryl group having 6 to 12 carbon atoms, and each * represents a bonding position.)

### (Benzoxazine Compound Represented by General Formula (1))

Each R₁ in general formula (1) is independently preferably a hydrogen atom or an alkyl group having 1 to 4 carbon atoms, more preferably a hydrogen atom or an alkyl group having one carbon atom (a methyl group), particularly preferably a hydrogen atom. When R₁ is not a hydrogen atom, the bonding position thereof is preferably the ortho position on the benzene ring with respect to the oxygen atom of each benzoxazine ring in general formula (1).

Each R₂ in general formula (1) is independently a divalent group having 1 to 10 carbon atoms, and specific examples include linear alkylene groups having 1 to 10 carbon atoms such as a methylene group, an ethylene group, a propane-1,2-diyl group, a propane-1,3-diyl group, a butane-1,4-diyl group, a pentane-1,5-diyl group, a hexane-1,6-diyl group, a cyclohexane-1,3-diyl group, and a cyclohexane-1,4-diyl group, branched alkylene groups having 3 to 10 carbon atoms, cyclic alkane-containing alkylene groups having 3 to 10 carbon atoms, alkylidene groups having 2 to 10 carbon atoms such as an ethylidene group, a propylidene group, an isopropylidene group, a butylidene group, a cyclopentylidene group, and a cyclohexylidene group, a phenylene group, and benzene ring-containing divalent groups having 6 to 10 carbon atoms such as groups represented by the following formulas.

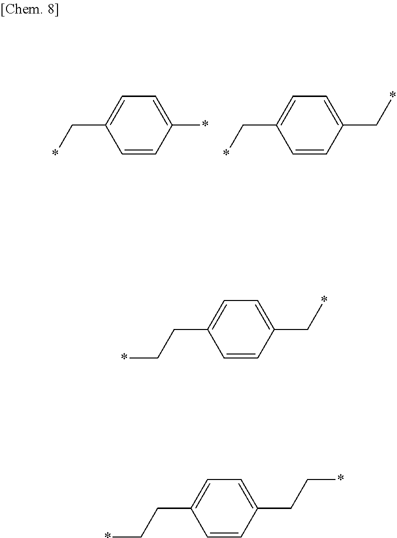

(In the formulas, * represents a bonding position.)

Of these, R₂ is preferably a linear alkylene group having 1 to 10 carbon atoms, a branched alkylene group having 3 to 10 carbon atoms, a cyclic alkane-containing alkylene group having 3 to 10 carbon atoms, or an alkylidene group having 2 to 10 carbon atoms, more preferably a linear alkylene group having 1 to 10 carbon atoms, a branched alkylene group having 3 to 10 carbon atoms, or a cyclic alkane-containing alkylene group having 3 to 10 carbon atoms, still more preferably a linear alkylene group having 1 to 6 carbon atoms, a branched alkylene group having 3 to 6 carbon atoms, or a cyclic alkane-containing alkylene group having 6 to 10 carbon atoms, particularly preferably a linear alkylene group having 1 to 4 carbon atoms or a branched alkylene group having 3 to 6 carbon atoms.

X in general formula (1) is preferably a single bond, a divalent group represented by general formula (1a), or a divalent group represented by (1b), more preferably a single bond or a divalent group represented by general formula (1a), particularly preferably a divalent group represented by general formula (1a).

When X in general formula (1) is represented by general formula (1a), R₃ and R₄, independently of each other, are more preferably a hydrogen atom, an alkyl group having 1 to 6 carbon atoms, an alkyl halide group having 1 to 6 carbon atoms, or an aryl group having 6 to 12 carbon atoms, still more preferably a hydrogen atom or an alkyl group having 1 to 4 carbon atoms, particularly preferably a hydrogen atom, a methyl group, or an ethyl group.

R₃ and R₄ may be bonded to each other to together form a cycloalkylidene group having 5 to 20 carbon atoms. The cycloalkylidene group having 5 to 20 carbon atoms may include a branched-chain alkyl group. The cycloalkylidene group preferably has 5 to 15 carbon atoms, more preferably has 6 to 12 carbon atoms, and particularly preferably has 6 to 9 carbon atoms.

Specific examples of the cycloalkylidene group include a cyclopentylidene group (5 carbon atoms), a cyclohexylidene group (6 carbon atoms), a 3-methylcyclohexylidene group (7 carbon atoms), a 4-methylcyclohexylidene group (7 carbon atoms), a 3,3,5-trimethylcyclohexylidene group (9 carbon atoms), a cycloheptylidene group (7 carbon atoms), a bicyclo[2.2.1]heptane-2,2-diyl group (7 carbon atoms), a 1,7,7-trimethylbicyclo[2.2.1]heptane-2,2-diyl group (10 carbon atoms), a 4,7,7-trimethylbicyclo[2.2.1]heptane-2,2-diyl group (10 carbon atoms), a tricyclo[5.2.1.0^{2,6}]decane-8,8-diyl group (10 carbon atoms), a 2,2-adamantylidene group (10 carbon atoms), and a cyclododecanylidene group (12 carbon atoms). Preferred are a cyclohexylidene group (6 carbon atoms), a 3-methylcyclohexylidene group (7 carbon atoms), a 4-methylcyclohexylidene group (7 carbon atoms), a 3,3,5-trimethylcyclohexylidene group (9 carbon atoms), and a cyclododecanylidene group (12 carbon atoms), more preferred are a cyclohexylidene group (6 carbon atoms), a 3,3,5-trimethylcyclohexylidene group (9 carbon atoms), and a cyclododecanylidene group (12 carbon atoms), and particularly preferred are a cyclohexylidene group (6 carbon atoms) and a 3,3,5-trimethylcyclohexylidene group (9 carbon atoms).

When X in general formula (1) is represented by general formula (1b), Ar₁ and Ar₂ are preferably each independently a benzene ring or a naphthalene ring, and Ar₁ and Ar₂ are more preferably both benzene rings. For example, when Ar₁ and Ar₂ are both benzene rings, the group represented by general formula (1b) is a fluorenylidene group.

The position of bonding of X in general formula (1) to the two benzoxazine rings is preferably the ortho or para position on the benzene ring with respect to the oxygen atom of each benzoxazine ring.

Specific examples of the benzoxazine compound represented by general formula (1) include compounds (1-1) to (1-6) having the following chemical structures. The benzoxazine compound represented by general formula (1) is preferably at least one compound selected from these compounds (1-1) to (1-6).

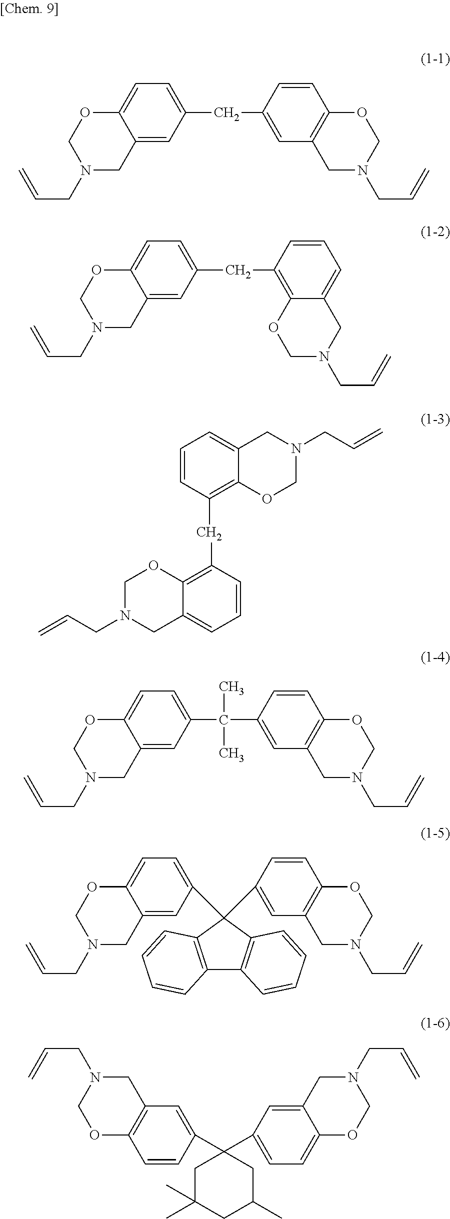

### (Ester Solvent Having Boiling Point at 1 Atm in Range of 30 °C or Higher and 90 °C or Lower)

Specific examples of the ester solvent having a boiling point at 1 atm in the range of 30 °C or higher and 90 °C or lower include methyl formate (32 °C), ethyl formate (54 °C), methyl acetate (57 °C), ethyl acetate (77 °C), methyl propionate (80 °C), and isopropyl acetate (88 °C). The temperatures given in parentheses are boiling points at 1 atm. Of these, methyl acetate, ethyl acetate, methyl propionate, and isopropyl acetate are preferred, ethyl acetate and methyl propionate are more preferred, and ethyl acetate is particularly preferred.

### (Solution)

The method of producing the benzoxazine solution containing the benzoxazine compound represented by general formula (1) and the ester solvent having a boiling point at 1 atm in the range of 30 °C or higher and 90 °C or lower is not particularly limited. Examples of such a production method include a production method in which the benzoxazine compound represented by general formula (1) is mixed with and dissolved in the ester solvent having a boiling point at 1 atm in the range of 30 °C or higher and 90 °C or lower, and a production method including a reaction step of performing a reaction in the presence of a bisphenol compound represented by general formula (2), a formaldehyde, an amine compound represented by general formula (3), and the ester solvent having a boiling point at 1 atm in the range of 30 °C or higher and 90 °C or lower, as described later.

The amount of the ester solvent having a boiling point at 1 atm in the range of 30 °C or higher and 90 °C or lower relative to the amount of the benzoxazine compound represented by general formula (1) in the benzoxazine solution is not particularly limited. In the benzoxazine solution for use in the solvent distilling-off step, the benzoxazine compound may be completely dissolved in the ester solvent or may be partially present in the form of a solid, an oily substance, liquid droplets, or the like.

### (Solvent Distilling-Off Step)

The solvent distilling-off step in the present invention is a step of subjecting the benzoxazine solution containing the benzoxazine compound represented by general formula (1) and the ester solvent having a boiling point at 1 atm in the range of 30 °C or higher and 90 °C or lower to removal of the solvent by distillation in the range of 20 °C or higher and 85 °C or lower.

The temperature range in the solvent distilling-off step is in the range of 20 °C or higher and 85 °C or lower, preferably in the range of 30 °C or higher and 55 °C or lower, more preferably in the range of 30 °C or higher and 50 °C or lower, particularly preferably in the range of 30 °C or higher and 45 °C or lower. As long as the solvent can be distilled off within the above temperature range, the pressure may be normal pressure or reduced pressure. It is preferable to perform the solvent distilling-off step under reduced pressure because the temperature is easily controlled to be in the above temperature range and the solvent can be distilled off at a lower temperature. The pressure in the case where the solvent distilling-off is performed under reduced pressure may be specifically, for example, in the range of 0.1 kPa or more and 95 kPa or less, and is more preferably in the range of 0.1 kPa or more and 70 kPa or less, still more preferably in the range of 0.1 kPa or more and 50 kPa or less. By removing the ester solvent from the benzoxazine solution by distillation in such a temperature range, the formation of high-molecular-weight components is suppressed to suppress a decrease in purity of the benzoxazine compound in the solvent distilling-off step, and an allyl group-containing benzoxazine compound with improved purity can be produced.

Depending on the process and handling after the solvent distilling-off step, the amount of the solvent to be removed can be adjusted to thereby adjust the amount of the solvent contained in the benzoxazine compound that has been subjected to removal of the solvent by distillation in the solvent distilling-off step. For example, when the benzoxazine compound is taken out from a vessel in which the solvent distilling-off step has been performed, the ester solvent may be hardly contained, specifically, for example, the content of the ester solvent relative to the amount of the benzoxazine compound obtained through the solvent distilling-off step may be 5 wt % or less, preferably 3 wt % or less, more preferably 1.5 wt % or less, still more preferably 1.0 wt % or less. To increase the concentration of the benzoxazine compound in the benzoxazine solution, the solvent can be removed by the solvent distilling-off step to a concentration at which the benzoxazine compound does not precipitate. Alternatively, the ester solvent can be removed from the benzoxazine solution by distillation in the solvent distilling-off step such that a solid of the benzoxazine compound is precipitated to form a slurry.

### <Reaction Step>

The benzoxazine solution containing the benzoxazine compound represented by general formula (1) and the ester solvent having a boiling point at 1 atm in the range of 30 °C or higher and 90 °C or lower, on which the solvent distilling-off step according to the present invention is performed, can be produced through a reaction step of performing a reaction in the presence of a bisphenol compound represented by general formula (2), a formaldehyde, an amine compound represented by general formula (3), and the ester solvent having a boiling point at 1 atm in the range of 30 °C or higher and 90 °C or lower.

### (Bisphenol Compound Represented by General Formula (2))

The bisphenol compound used in the reaction step is a compound represented by general formula (2).

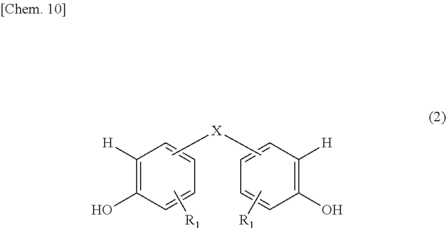

(In the formula, R₁ and X are as defined in general formula (1).)

The definition and preferred forms of R₁ in general formula (2) are the same as those in general formula (1). When R₁ in general formula (2) is not a hydrogen atom, the bonding position thereof is preferably the ortho position on the benzene ring with respect to the hydroxy group of each benzene ring in general formula (2).

The definition and preferred forms of X in general formula (2) are the same. The position of bonding of X in general formula (2) to the two benzoxazine rings is preferably the ortho or para position with respect to each hydroxy group.

Specific examples of the bisphenol compound represented by general formula (2) include bisphenol F (bis(2-hydroxyphenyl)methane, 2-hydroxyphenyl-4-hydroxyphenylmethane, bis(4-hydroxyphenyl)methane), bisphenol A (2,2-bis(4-hydroxyphenyl)propane), bisphenol C (2,2-bis(4-hydroxy-3-methylphenyl)propane), bisphenol Z (1,1-bis(4-hydroxyphenyl)cyclohexane), bisphenol TMC (1,1-bis(4-hydroxyphenyl)-3,3,5-trimethylcyclohexane), and 9,9-bis(4-hydroxyphenyl)fluorene. The bisphenol compound represented by general formula (2) is preferably at least one compound selected from these compounds listed as specific examples, more preferably at least one compound selected from bisphenol F (bis(2-hydroxyphenyl)methane, 2-hydroxyphenyl-4-hydroxyphenylmethane, bis(4-hydroxyphenyl)methane), bisphenol A (2,2-bis(4-hydroxyphenyl)propane), bisphenol TMC (1,1-bis(4-hydroxyphenyl)-3,3,5-trimethylcyclohexane), and 9,9-bis(4-hydroxyphenyl)fluorene. Bisphenol F (bis(2-hydroxyphenyl)methane, 2-hydroxyphenyl-4-hydroxyphenylmethane, and bis(4-hydroxyphenyl)methane) may be a mixture of these compounds, and such a mixture may be used.

### (Formaldehyde)

Specific examples of the formaldehyde used in the reaction step include an aqueous formaldehyde solution, 1,3,5-trioxane, and paraformaldehyde.

### (Amine Compound)

The amine compound used in the reaction step is a compound represented by general formula (3).

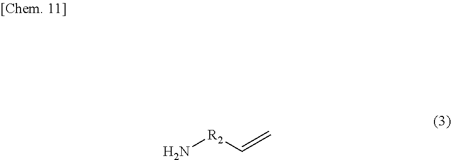

(In the formula, R₂ is as defined in general formula (1).)

The definition and preferred forms of R₂ in general formula (2) are the same as those in general formula (1).

Specific examples of the amine compound represented by general formula (3) include allylamine, 4-allylaniline, 3-butene-1-amine, 4-pentene-1-amine, and 4-ethenylcyclohexanamine. Allylamine is particularly preferred.

The amine compound can also be used in the form of a salt formed with an inorganic acid such as hydrochloric acid or sulfuric acid, and in the reaction step in this case, the reaction is carried out in a state where an alkaline aqueous solution obtained by dissolving sodium hydroxide, potassium hydroxide, sodium carbonate, potassium carbonate, sodium hydrogen carbonate, potassium hydrogen carbonate, or the like in water is further present.

### (Catalyst)

In the reaction step, a catalyst for accelerating the reaction is not particularly necessary, but an acid catalyst or a base catalyst can be used as needed. In this case, examples of acid catalysts that can be used include, but are not limited to, concentrated hydrochloric acid, hydrochloric acid gas, trifluoroacetic acid, methanesulfonic acid, p-toluenesulfonic acid, benzoic acid, and mixtures thereof, and examples of base catalysts that can be used include, but are not limited to, sodium hydroxide, sodium carbonate, triethylamine, triethanolamine, and mixtures thereof.

### (Amount Used)

In the reaction step, the amount of the formaldehyde used is preferably in the range of 4.0 to 20.0 mol, more preferably in the range of 4.0 to 16.0 mol, still more preferably in the range of 4.0 to 12.0 mol, relative to 1 mol of the bisphenol compound represented by general formula (2).

The amount of the amine compound represented by general formula (3) used is preferably in the range of 2.0 to 10.0 mol, more preferably in the range of 2.0 to 8.0 mol, still more preferably in the range of 2.0 to 6.0 mol, relative to 1 mol of the bisphenol compound represented by general formula (2).

The amount of the ester solvent having a boiling point at 1 atm in the range of 30 °C or higher and 90 °C or lower used is not particularly limited as long as the reaction is not hindered, but is preferably in the range of 0.5 to 5 times, more preferably in the range of 1 to 3 times the amount of the bisphenol compound represented by general formula (2) on a weight basis.

In addition to the ester solvent having a boiling point at 1 atm in the range of 30 °C or higher and 90 °C or lower, other solvents can be used in combination as long as the solvents do not inhibit the reaction and the types and amounts of the solvents used allow the solvent distilling-off step to be performed in the above-mentioned temperature range. Examples of the types of solvents that can be used include toluene, xylene, chloroform, dichloromethane, tetrahydrofuran, and dioxane.

### (Reaction Conditions)

The reaction temperature in the reaction step is in the range of 10 °C to 80 °C, preferably in the range of 20 °C to 60 °C, more preferably in the range of 20 °C to 50 °C, particularly preferably in the range of 30 °C to 50 °C.

The reaction may be carried out under normal pressure conditions, or may be carried out under increased pressure or reduced pressure.

The method of mixing the bisphenol compound represented by general formula (2), the formaldehyde, and the amine compound represented by general formula (3) used as raw materials is not particularly limited. Examples include (a) a method in which a mixture containing the bisphenol compound represented by general formula (2) and the formaldehyde is mixed with the amine compound represented by general formula (3) to carry out the reaction and (b) a method in which a mixture containing the formaldehyde and the amine compound represented by general formula (3) is mixed with the bisphenol compound represented by general formula (2). These mixtures may contain the solvent and the catalyst described above. The method of mixing the catalyst is also not particularly limited, but the catalyst is preferably mixed before the amine compound represented by general formula (3) is mixed.

In the production method according to the present invention, the method of mixing a mixture of raw materials with the rest of the raw materials is not particularly limited, but from the viewpoint of reaction selectivity and suppression of formation of by-product high-molecular-weight components, continuous or intermittent mixing for, for example, 10 minutes to 2 hours is preferred to mixing all at once.

In another embodiment, a process of removing water derived from the raw materials or water generated during the reaction out of the system may be included. The process of removing water generated from a reaction liquid is not particularly limited and can be performed by distilling the generated water azeotropically with the solvent system in the reaction liquid. The generated water can be removed out of the reaction system by using, for example, an isobaric dropping funnel equipped with a cock, a Dimroth condenser, or a Dean-Stark apparatus.

The benzoxazine solution obtained through the reaction step may be washed by a washing step of performing washing with water or an alkaline aqueous solution obtained by dissolving sodium hydroxide, potassium hydroxide, sodium carbonate, potassium carbonate, sodium hydrogen carbonate, potassium hydrogen carbonate, or the like in water. After the washing with an alkaline aqueous solution, washing with water is preferably performed so that the alkali does not remain in the reaction liquid. Since the ester solvent having a boiling point at 1 atm in the range of 30 °C or higher and 90 °C or lower is used in the reaction step, the resulting reaction liquid is separable from water, so that the washing step can be performed subsequently to the reaction step, and the benzoxazine compound can be produced through an efficient process.

The solution obtained through the reaction step may contain a salt and unreacted paraformaldehyde, and a filtration step of removing them by filtration may be performed.

When the benzoxazine compound represented by general formula (1) obtained by performing the solvent distilling-off step is taken out from a vessel in which the solvent distilling-off step has been performed, it can be taken out as it is in the case of a benzoxazine compound that is in a flowable state at room temperature, or after being brought into a flowable state by heating in the case of a benzoxazine compound that is solid at room temperature, or the benzoxazine compound can be taken out in such a manner that it is precipitated by being added to a poor solvent or it is crystallized by adding a solvent to the reaction mixture and filtered. The solid of the benzoxazine compound separated by filtration is preferably dried to remove the solvent contained in the solid. The benzoxazine compound taken out by any of these methods may be further made into a high-purity product by, for example, standard purification means such as washing with a solvent or water or recrystallization.

### Another Embodiment of Present Invention

Another embodiment of the present invention is, for example, a production method including a reaction step of obtaining a benzoxazine solution containing a benzoxazine compound represented by general formula (1) and an ester solvent having a boiling point at 1 atm in the range of 30 °C or higher and 90 °C or lower, and a solvent distilling-off step of subjecting the solution obtained in the reaction step to removal of the solvent by distillation in the range of 20 °C or higher and 85 °C or lower. The production method may further include the washing step and/or the filtration step between the reaction step and the solvent distilling-off step.

### EXAMPLES

The present invention will now be described more specifically with reference to Examples.

### <Analysis Method>

### 1. Reaction Liquid Composition and Purity Analysis (Gel Permeation Chromatography: GPC)

The purity of benzoxazine compounds synthesized by the production method according to the present invention was defined as the area percentage of the benzoxazine compounds determined by this analysis.

Apparatus: HLC-8320/manufactured by Tosoh Corporation

Detector: differential refractometer (RI)

### [Measurement Conditions]

Flow rate: 1 mL/min.

Eluate: tetrahydrofuran

Temperature: 40 °C

Wavelength: 254 nm

Measurement sample: One gram of a benzoxazine compound-containing composition was 200-fold diluted with tetrahydrofuran.

### Example 1

In a 500 mL four-necked flask equipped with a thermometer, a stirrer, and a condenser, 19.5 g of water and 19.5 g of NaOH (granular) were loaded and stirred. To this alkaline solution, 19.5 g of allylamine hydrochloride was added, and stirring was performed for 1 hour in a nitrogen atmosphere. Thereafter, 39.1 g of paraformaldehyde (purity: 92%) was added portionwise, and stirring was performed for 5 hours. To this solution, 93 g of ethyl acetate and 50 g of bisphenol F were added, and stirring was performed at 30 °C to 40 °C for 13 hours. Since the viscosity increased during the stirring, 46 g of ethyl acetate was added. The reaction liquid was analyzed by high-performance liquid chromatography (HPLC), as a result of which disappearance of bisphenol F was confirmed. The reaction liquid was analyzed by GPC, revealing that the proportion of the benzoxazine compound present in the reaction liquid was 65 area %, with the remaining 35 area % being accounted for by compounds having molecular weights higher than that of the benzoxazine compound, that is, high-molecular-weight components.

After completion of the reaction, salts and unreacted paraformaldehyde were removed by filtration. The filtrate was washed five times with 50 mL of water.

The washed filtrate was subjected to distillation at 40 °C under reduced pressure to remove the solvent. The pressure during the distillation was gradually reduced so as to finally reach 1.4 kPa.

After the solvent was distilled off, the residue was cooled to obtain 55 g of the benzoxazine compound as a flowable oily substance. The amount of solvent contained in the oily substance was 1.0 wt %. The obtained oily substance was measured by GPC under the above analysis conditions, revealing that the purity was 61 area %, and the content of the high-molecular-weight components was 39 area %.

### Comparative Example 1

In a 500 mL four-necked flask equipped with a thermometer, a stirrer, and a condenser, 19.5 g of water and 19.5 g of NaOH (granular) were loaded and stirred. To this alkaline solution, 19.5 g of allylamine hydrochloride was added, and stirring was performed for 1 hour in a nitrogen atmosphere. Thereafter, 39.1 g of paraformaldehyde (purity: 92%) was added portionwise, and stirring was performed for 5 hours. To this solution, 93 g of toluene and 50 g of bisphenol F were added, and stirring was performed at 90 °C for 1.5 hours. The reaction liquid was analyzed by HPLC, as a result of which disappearance of bisphenol F was confirmed. The reaction liquid was analyzed by GPC, revealing that the proportion of the benzoxazine compound present in the reaction liquid was 38 area %, with the remaining 62 area % being accounted for by high-molecular-weight components.

After completion of the reaction, salts and unreacted paraformaldehyde were removed by filtration. The filtrate was washed five times with 50 mL of water.

The washed filtrate was subjected to distillation at 60 °C to 90 °C under reduced pressure to remove the solvent. The pressure during the distillation was gradually reduced so as to finally reach 1.4 kPa.

After the solvent was distilled off, the residue was cooled to obtain a benzoxazine compound as a non-flowable solid substance. The amount of solvent contained in the solid substance was 1.0 wt %. The obtained benzoxazine compound was measured by GPC under the above analysis conditions, revealing that the purity was 32 area %, and the content of the high-molecular-weight components was 68 area %.

### Example 2

To a 2 L four-necked flask equipped with a thermometer, a stirrer, a condenser, and a dropping funnel, 130 g (1.3 moL) of allylamine hydrochloride was added, and then 110 g (1.3 moL) of a 48% aqueous NaOH solution was slowly added over 5 minutes with stirring while the rise of temperature was monitored. Thereafter, after confirming that the pH of the aqueous layer was about 9 to 10, 107 g (3.3 moL) of paraformaldehyde (purity: 92%) was added portionwise over 30 minutes. In this process, the temperature of the reaction system was observed to increase from 30 °C to 55 °C Thereafter, the mixture was cooled in air with stirring, and after confirming that the temperature had decreased to 30 °C, stirring was performed at 30 °C for 1 hour.

After completion of the stirring, 586 g of ethyl acetate and 210 g of 1,1-bis(4-hydroxyphenyl)-3,3,5-trimethylcyclohexane were added into the flask. Thereafter, the temperature of the liquid in the flask was increased to 55 °C, and the reaction was carried out at 55 °C for 24 hours, at 60 °C for 3 hours, at 65 °C for 5 hours, and at 70 °C for 1 hour. The reaction liquid was analyzed by HPLC, as a result of which disappearance of 1,1-bis(4-hydroxyphenyl)-3,3,5-trimethylcyclohexane was confirmed. After completion of the reaction, the temperature of the liquid in the flask was lowered to 40 °C. The reaction liquid was analyzed by GPC, revealing that the proportion of the benzoxazine compound present in the reaction liquid was 75 area %, with the remaining 25 area % being accounted for by high-molecular-weight components.

After the final reaction liquid was mixed with 400 g of pure water, the mixture was stirred for 30 minutes and allowed to stand, and after confirming separation from an organic layer, an aqueous layer was removed. This water-washing operation was performed six times, and it was confirmed that the pH of the aqueous layer was 7 to 8.

Thereafter, the solvent was removed by distillation at 40 °C under reduced pressure. After the solvent was distilled off, the residue was cooled to obtain a benzoxazine compound as a non-flowable solid. The obtained benzoxazine compound was measured by GPC under the above analysis conditions, revealing that the purity was 70 area %, and the content of the high-molecular-weight components was 30 area %.

The obtained distillation residue was heated to 90 °C, poured out on a metal tray, cooled to room temperature, and crushed to obtain 250 g of a benzoxazine compound as a yellow solid. The amount of solvent contained in the solid was 1.0 wt %. The yield was 78 mol % based on the 1,1-bis(4-hydroxyphenyl)-3,3,5-trimethylcyclohexane used.

The obtained solid substance was subjected to ¹H-NMR analysis; peaks attributed to a 3,3,5-trimethylcyclohexylidene group were observed near 0.4 ppm and 0.8 to 1.0 ppm, a peak attributed to a benzoxazine moiety was observed near 3.3 to 4.0 ppm, a peak attributed to allylamine was observed at 4.8 to 5.2 ppm, and a peak attributed to an aromatic was observed near 6.6 to 7.3 ppm. The results of the analysis revealed that the solid substance was a benzoxazine compound, that is, the target compound.

For the benzoxazine compound obtained in Comparative Example 1, which was not produced by the production method according to the present invention, and the benzoxazine compounds obtained in Examples 1 and 2, which were produced by the production method according to the present invention, the purities of the benzoxazine compounds before and after the solvent distilling-off step and their degree of change are listed in Table 1.

TABLE 1

| | Solvent distilling- | Purity | Degree of |
|---|---|---|---|
| | off step | (area %) | change (%) |
| Example 1 | before | 65 | −6.2 |
| | after | 61 | |
| Comparative | before | 38 | −15.8 |
| Example 1 | after | 32 | |
| Example 2 | before | 75 | −6.7 |
| | after | 70 | |

From these results, it has become clear that the production method according to the present invention can suppress a decrease in purity in the solvent distilling-off step and can produce a benzoxazine compound with improved purity.

1. A method for producing a benzoxazine compound represented by general formula (1), comprising a solvent distilling-off step of subjecting a benzoxazine solution containing the benzoxazine compound represented by general formula (1) and an ester solvent having a boiling point at 1 atm in a range of 30 °C or higher and 90 °C or lower to removal of the solvent by distillation in a range of 20 °C or higher and 85 °C or lower: 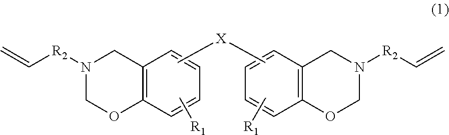
where each R₁ independently represents a hydrogen atom or an alkyl group having 1 to 6 carbon atoms, each R₂ independently represents a divalent hydrocarbon group having 1 to 10 carbon atoms, and X represents a single bond, an oxygen atom, a sulfur atom, a sulfonyl group, a carbonyl group, a divalent group represented by general formula (1a), or a divalent group represented by (1b); 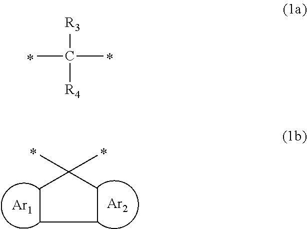
where R₃ and R₄ each independently represent a hydrogen atom, an alkyl group having 1 to 10 carbon atoms, an alkyl halide group having 1 to 10 carbon atoms, or an aryl group having 6 to 12 carbon atoms, R₃ and R₄ may be bonded to each other to together form a cycloalkylidene group having 5 to 20 carbon atoms, Ar₁ and Ar₂ each independently represent an aryl group having 6 to 12 carbon atoms, and each * represents a bonding position.

2. The production method according to claim 1, wherein the ester solvent is ethyl acetate.

3. The production method according to claim 1, wherein the benzoxazine compound represented by general formula (1) is at least one compound selected from compounds (1-1) to (1-6):

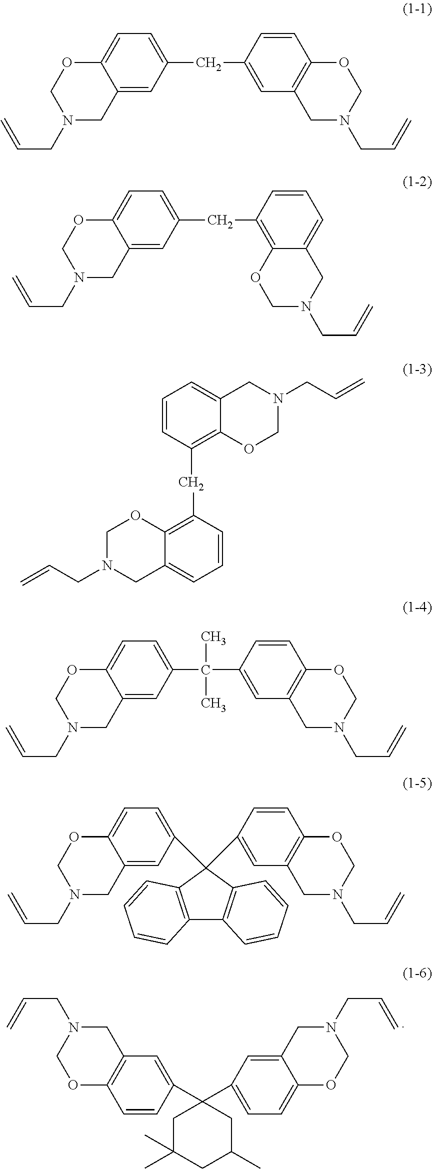

4. The production method according to claim 1, wherein the benzoxazine solution is a benzoxazine solution obtained through a reaction step of performing a reaction in the presence of a bisphenol compound represented by general formula (2), a formaldehyde, an amine compound represented by general formula (3), and the ester solvent having a boiling point at 1 atm in the range of 30 °C or higher and 90 °C or lower: 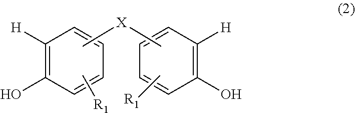
where R₁ and X are as defined in general formula (1); 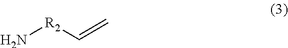
where R₂ is as defined in general formula (1).

5. The production method according to claim 4, wherein the bisphenol compound represented by general formula (2) is at least one compound selected from bisphenol F (bis(2-hydroxyphenyl)methane, 2-hydroxyphenyl-4-hydroxyphenylmethane, bis(4-hydroxyphenyl)methane), bisphenol A (2,2-bis(4-hydroxyphenyl)propane), bisphenol C (2,2-bis(4-hydroxy-3-methylphenyl)propane), bisphenol Z (1,1-bis(4-hydroxyphenyl)cyclohexane), bisphenol TMC (1,1-bis(4-hydroxyphenyl)-3,3,5-trimethylcyclohexane), and 9,9-bis(4-hydroxyphenyl)fluorene, and the amine compound represented by general formula (3) is allylamine.
